(19) United States
(12) Patent Application Publication
(10) Pub. No.: US 2021/0353805 A1
(43) Pub. Date: Nov. 18, 2021

(54) PORTABLE DISINFECTION UNITS FOR OFFICE WORKSTATIONS

(71) Applicant: Sylvan R. Shemitz Designs, LLC, West Haven, CT (US)

(72) Inventor: David R. PFUND, Woodbridge, CT (US)

(21) Appl. No.: 17/315,566

(22) Filed: May 10, 2021

Related U.S. Application Data
(60) Provisional application No. 63/023,472, filed on May 12, 2020.

(51) Int. Cl. A61L 2/24; A61L 2/10

(52) U.S. Cl. CPC A61L 2/24; A61L 2202/25; A61L 2202/14; A61L 2/10

(57) ABSTRACT

A portable disinfection device includes at least one disinfection energy source and a control circuit configured to activate and deactivate the disinfection energy source. A communications means is provided to allow the control circuit to activate and deactivate the disinfection energy source in response to a remote controller. The control circuit is further configured with a countdown timer to automatically deactivate the disinfection energy source after a preset period of operation. A further portable disinfection device includes a light source, and operator interface for controlling the light source, and an operator interface for activating and deactivating the disinfection energy source.

### CROSS REFERENCE TO RELATED APPLICATION

This application is related to and claims the benefit of U.S. Provisional Patent Application Ser. No. 63/023,472 filed on May 12, 2020, the entire contents of which are incorporated herein by reference.

### TECHNICAL FIELD

The disclosure relates to a disinfection device for an office. More particularly, the disclosure relates to a portable disinfection device for an office workstation and for use in conjunction with a remote controller of said office, said portable disinfection device comprising a control circuit configured for communication with said remote controller whereby said controller may be used to activate and deactivate a disinfection energy source of said disinfection device.

### BACKGROUND

Pathogens exist on workstation surfaces and in air in offices. Offices require disinfection of air and workstation surfaces such as desk/work surfaces and the surfaces of sneeze shields provided to curtail the spread of pathogens between workstations. Fixtures installed in buildings use disinfecting energy sources (e.g. sources that emit UVC ultraviolet energy) to disinfect air, and, to minimize exposure to office occupants, may use disinfecting energy sources when offices are vacant to disinfect workstations surfaces. Furthermore, offices are commonly outfitted with controls for office environmental systems and/or security controls that monitor office occupancy and vacancy and provide for corresponding automatic and/or manual control of office environmental systems such as lighting, heating and air conditioning. Accordingly, these controls can be used to safely operate systems and devices that emit disinfection energy in office spaces and at workstations. However, the effectiveness of fixed and permanently installed disinfection energy systems for disinfecting workstations surfaces is adversely impacted by their lack of proximity to, and alignment with, said workstations surfaces and by shadowing cause by sneeze shields and the like, especially at small workstations. Moreover, small workstations suffer from a lack of space to implement local disinfecting units in addition to other workstation accessories and work tools such as ambient lights, supplemental task lights, computer monitors, and the like.

Accordingly, it is desirable that portable workstation disinfecting units be provided and configured for operation by common building automation control systems. It is further desirable that these workstation disinfection units provide a visual indication that disinfection energy emanations are, or are not, present in the office. It is further desirable that these workstation disinfection units also serve to provide task lighting at workstations for the performance of visual tasks by workstation occupants and/or to provide indirect general ambient lighting for the office.

### BRIEF SUMMARY

A portable disinfection device for workstations in an office is provided herein including at least one disinfection energy source and a control circuit configured to communicate with a control system of said office to allow for the activation and deactivate said disinfection energy source by said control system.

### BRIEF DESCRIPTION OF THE DRAWINGS

Referring to the exemplary drawings wherein like elements are numbered alike in the several FIGS.:

FIG. 1 is a diagram of an office comprising an exemplary embodiment of a disinfecting unit of the invention;

FIG. 2 is a diagram of an office comprising another exemplary embodiment of a disinfecting unit of the invention;

FIG. 3A is a diagram of a control circuit of the disinfecting unit in FIG. 1;

FIG. 3B is a diagram of a control circuit of the disinfecting unit in FIG. 2;

FIG. 3C is another diagram of a control circuit of the disinfecting unit in FIG. 2;

FIG. 3D is another diagram of a control circuit of the disinfecting unit in FIG. 2;

FIG. 4 is a diagram of an office comprising another exemplary embodiment of a disinfecting unit of the invention;

FIG. 5 is a diagram of a control circuit of the disinfecting unit in FIG. 4;

FIG. 6 is a diagram of an office comprising another exemplary embodiment of a disinfecting unit of the invention;

FIG. 7 is a diagram of an office comprising another exemplary embodiment of a disinfecting unit of the invention;

FIG. 8 is a diagram of an office comprising another exemplary embodiment of a disinfecting unit of the invention;

FIG. 9 is a diagram of an office comprising another exemplary embodiment of a disinfecting unit of the invention;

FIG. 10A is a diagram of a control circuit of the disinfecting units in FIGS. 6 and 8;

FIG. 10B is a diagram of a control circuit of the disinfecting units in FIGS. 7 and 9;

FIG. 11 is a diagram of an office comprising another exemplary embodiment of a disinfecting unit of the invention;

FIG. 12A is a diagram of a control circuit of the disinfecting unit in FIG. 11;

FIG. 12B is another diagram of a control circuit of the disinfecting unit in FIG. 11;

FIG. 13 is a diagram of an office comprising another exemplary embodiment of a disinfecting unit of the invention;

FIG. 14 is a diagram of a control circuit of the disinfecting unit in FIG. 13;

FIG. 15 is a diagram of an office comprising another exemplary embodiment of a disinfecting unit of the invention;

FIG. 16 is a diagram of a control circuit of the disinfecting unit in FIG. 15;

FIG. 17 is perspective view of another exemplary embodiment of a disinfecting unit of the invention; and

FIG. 18 is a diagram of an office comprising another exemplary embodiment of a disinfecting unit of the invention.

### DETAILED DESCRIPTION OF THE EMBODIMENTS

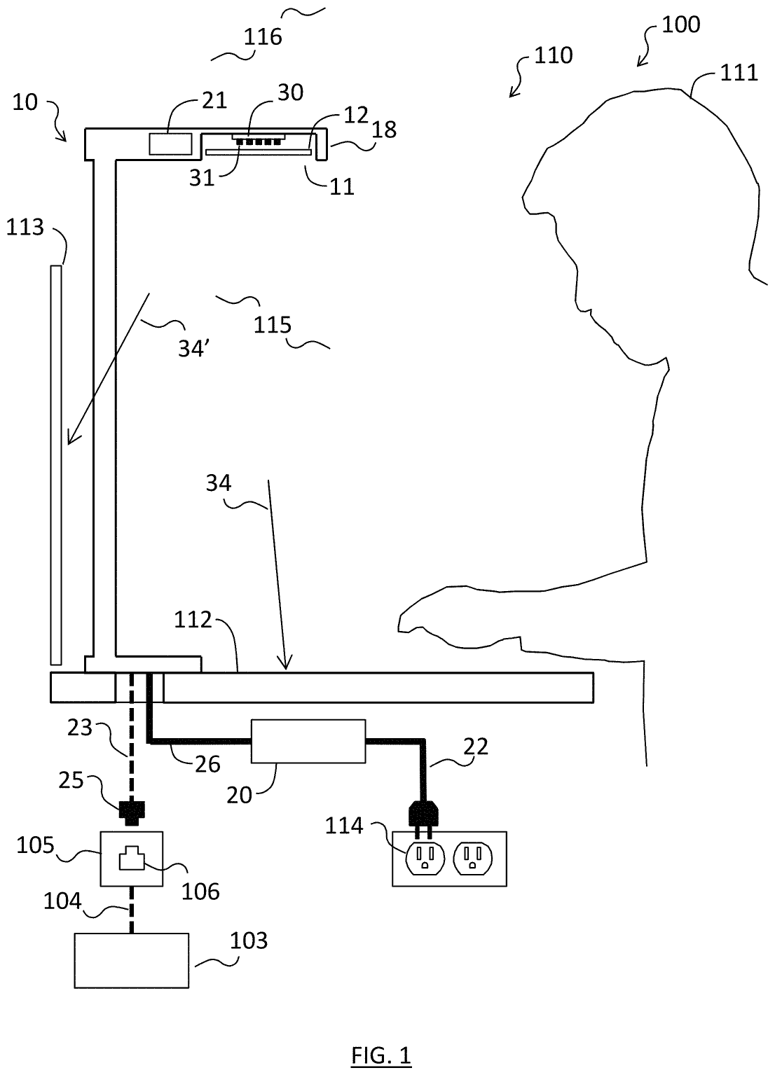

FIG. 1 discloses an office 100 with workstation 110, controller 103, and wiring terminal 105, said wiring terminal 105 comprising one or more communication port(s) 106, where wiring terminal 105 is disposed in proximity to workstation 110, and where wiring terminal 105 is further associated with controller 103 via control wiring 104, said controller 103 being configurable to automatically activate and deactivate interior environmental systems and/or system processes associated with office 100 according at least to the occupancy state of office 100 (occupied or unoccupied). Controller 103 may be located in office 100 or in a remote location, and may additionally or alternatively provide for manual activation and deactivation of interior environmental systems and/or system processes associated with office 100 when operated by authorized facility personnel. Said controller 103 may also comprise a configurable time clock for scheduling automatic activation and deactivation of interior environmental systems and/or system processes associated with office 100 during periods when office 100 is occupied and/or unoccupied.

Referring again to FIG. 1, workstation 110 is further comprised of work surface 112, sneeze barrier 113 and power receptacle 114.

Referring again to FIG. 1, in a embodiment, portable disinfection unit 10 is deployed in workstation 110, said disinfection unit 10 comprising device body 18, a disinfection energy source 31 disposed in or on device body 18, a control circuit 21 associated with disinfection energy source 31, power supply unit 20 associated with control circuit 21 via power cable 26 and associated with power receptacle 114 of workstation 110 via cord and plug 22. Disinfection device 10 may also comprise an emitter base in association with disinfection energy source 31. In this exemplary embodiment, control cable 23 is associated with control circuit 21, said control cable 23 being further associated with controller 103 via pluggable connector 25, communication port 106 of wiring terminal 105, and control wiring 104 such that control circuit 21 is in communication with controller 103; however, a direct wired connection between control cable 23 and control wiring 104 is also contemplated to achieve said communication. It is further conceived that power supply unit 20 may be associated with power cable 26 and/or power cord and plug 22 via one or more pluggable quick-connect/quick-disconnect connector(s) and port(s), that wiring terminal 105 and/or control circuit 21 may be disposed on or integral to power supply unit 20, and that either or both of power supply unit 20 and wiring terminal 105 may be disposed within or on device body 18 along with control circuit 21.

In this embodiment, disinfection energy source 31 is one or more LED(s) associated with emitter base 30, and lens 12 is a lens comprised of a material suitable for the transmission of electromagnetic energy of wavelengths emitted by disinfection energy source 31, whereby emitter base 30, disinfection energy source 31, and lens 12 are disposed in aperture 11 of device body 18 and configured such that disinfection emanations 34 and 34′ emitted by disinfection energy source 31 pass through lens 12 and are directed toward work surface 112 and sneeze barrier 113 of workstation 110. Furthermore, control circuit 21 is associated with emitter base 30 and disinfection energy source 31 and advantageously configured with a program to activate and deactivate disinfection energy source 31 in response to signals received from controller 103. Said program of control circuit 21 may be further configured to deactivate and prevent the operation of disinfection energy source 31 when control circuit 21 is not in communication with controller 103.

In this embodiment, lens 12 is configured with prismatic features to distribute disinfection emanations 34 and 34′ in a wide pattern to irradiate the surfaces of work surface 112 and sneeze barrier 113 exposed to lens 12. Accordingly, any workstation accessories, office supplies, and/or personal effects disposed on work surface 112 and sneeze barrier 113 are effectively irradiated likewise, as are any pathogens occurring on said surfaces, workstation accessories, office supplies, and/or personal effects. Similarly, any pathogens occurring in the air 115 circulating between lens 12 and said surfaces are likewise irradiated. Furthermore, in this embodiment, disinfection energy source 31 emits near-ultraviolet short-wave electromagnetic energy with a peak intensity wavelength measuring between 400 nm and 420 nm known to kill or otherwise inactivate certain pathogens and known to be harmless to humans. Similarly, in another embodiment, disinfection energy source 31 emits ultraviolet short-wave electromagnetic energy with a peak intensity wavelength measuring between 200 nm and 280 nm known to kill or otherwise inactivate other known pathogens. It is further conceived that the peak intensity emissions of disinfection energy source 31 may comprise other wavelengths of short-wave electromechanical energy Furthermore, it is conceived that aperture 11, lens 12 and emitter base 30 with disinfection energy source 31 may be inverted and rearranged and/or otherwise reoriented to instead kill or otherwise deactivate pathogens occurring in the air 116 circulating above workstation 110.

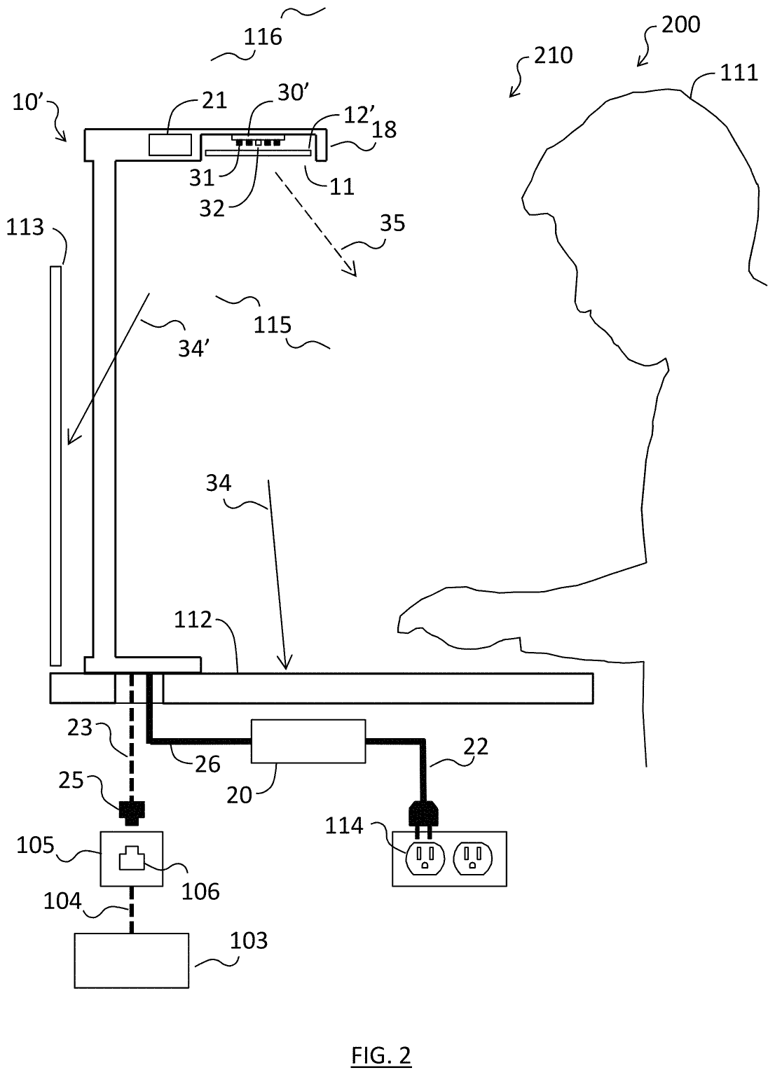

Referring now to FIG. 2, in another embodiment, where many of the items disclosed in office 100, workstation 110, and disinfection unit 10 of FIG. 1 are deployed in office 200, workstation 210, and disinfection unit 10′ disposed therein, lens 12′ is a lens comprised of a material suitable for the transmission of both light and electromagnetic energy of wavelengths emitted by disinfection energy source 31, disinfection energy source 31 is disposed on emitter base 31′, and light source 32 is disposed on emitter base 31′ such that emanations 35 from light source 32 pass through lens 12′ to generally provide for illumination of work surface 112 and other objects and surfaces occurring in workstation 210 and office 200 as seen by workstation occupant 111, other room occupants, and/or other persons viewing office 200 from any adjacent space. Accordingly, and in accordance with the present invention, light source 32 may be activated and deactivated in unison with disinfection energy source 31 and may further be one or more red LED(s) (i.e. an LED that emits light in the red region of the visual spectrum) whereby said illumination serves as a visual indicator that disinfection emanations 34 and 34′ are (or are not) present in office 200 and in workstation 210.

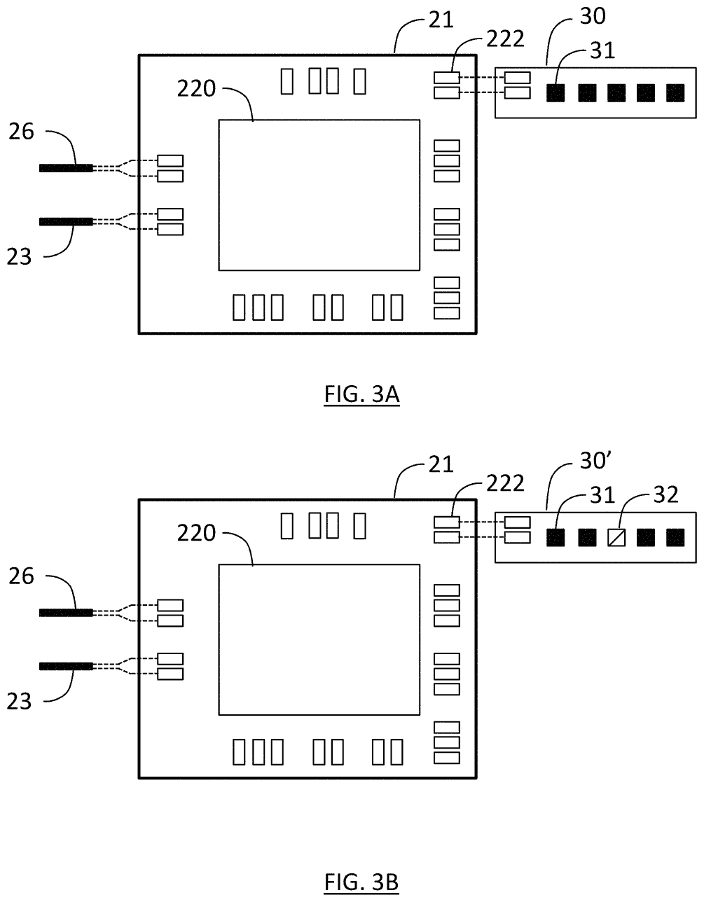

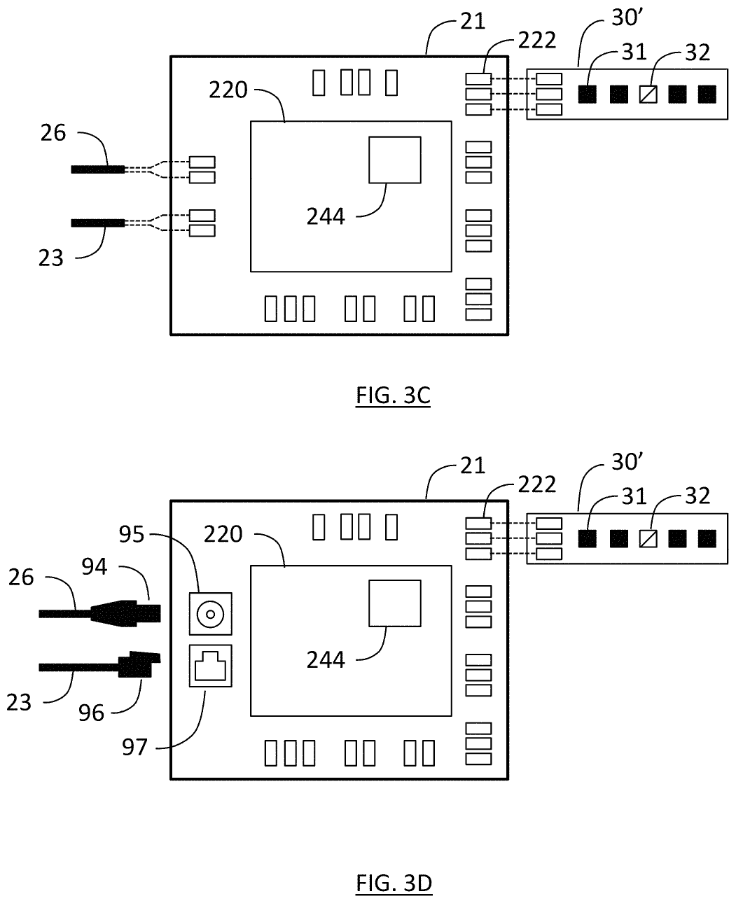

Referring now to FIGS. 3A and 3B, control circuit 21 of FIGS. 1 and 2 is further disclosed to comprise connection terminals 222 and I/O processor 220, whereby terminals 222 associate control circuit 21 with power cable 26 and control cable 23, and with disinfection energy source 31 and light source 32 disposed on emitter bases 30 and 30′, whereby I/O processor 220 executes the aforementioned program of control circuit 21 to activate and deactivate disinfection energy source 31 and light source 32 in response to signals received via control cable 23. As previously noted, said program may be further configured to deactivate and prevent the operation of disinfection energy source 31 when control circuit 21 is not in communication with controller 103 even though said control circuit 21 remains connected to a power source via power cable 26.

Referring now to FIG. 3C, where I/O processor 220 of control circuit 21 further comprises a timer circuit 244 and is configured with a program whereby control circuit 21 activates and deactivates disinfection energy source 31 disposed on emitter base 30′ in response to signals received via control cable 23 and separately activates and deactivates light source 32 disposed on emitter base intermittently according to a timer function of timer circuit 244 while disinfection energy source 31 is activated. This functionality provides further visual indication that a routine delivering disinfection emanations 34 and 34′ to office 200 and to workstation 210 is active (see FIG. 2).

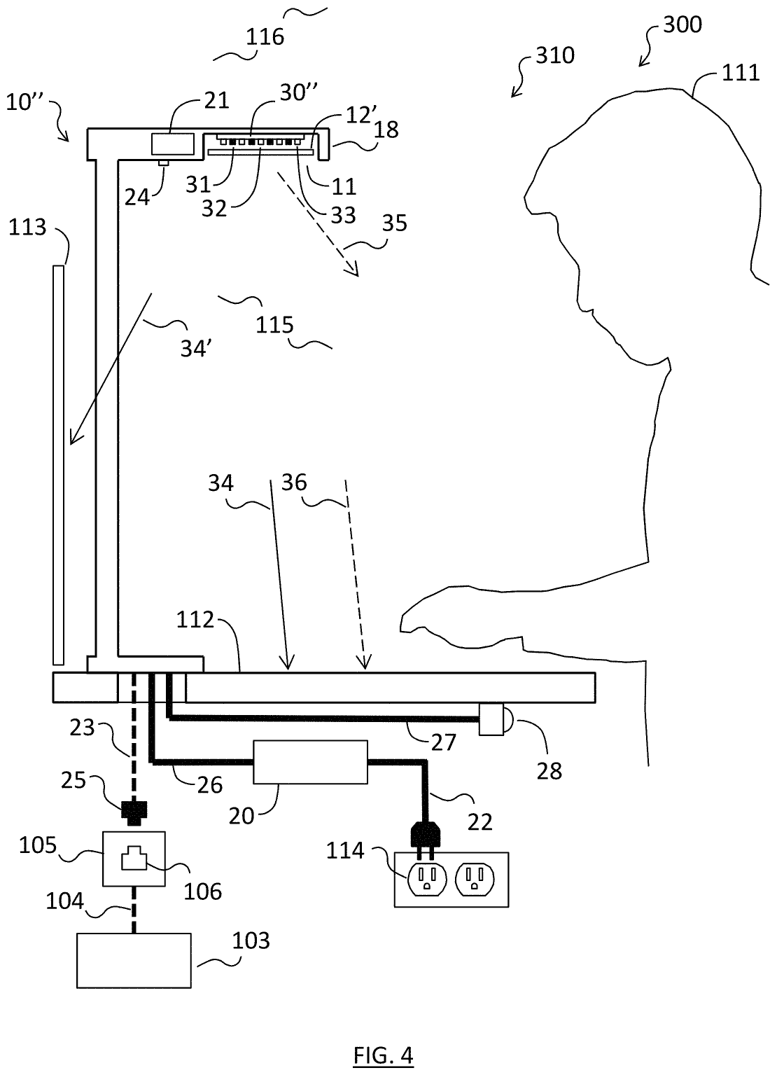

Referring now to FIG. 4, in another embodiment, where many of the items disclosed in office 200, workstation 210, and disinfection unit 10′ of FIG. 2 are deployed in office 300, workstation 310, and disinfection unit 10″ disposed therein, disinfection energy source 31 is disposed on emitter base 31″ and light source 33 is likewise disposed on emitter base 31″ such that emanations 36 from light source 33 pass through lens 12′ to provide for task illumination of work surface 112. Furthermore, and in accordance with the present invention, aforementioned light source 32 may also be disposed on emitter base 31″. In this and other embodiments, light source 33 may be one or more LED(s) that emit(s) broad spectrum white light suitable for aiding workstation occupants in performing visual tasks. Furthermore, operator interface 24 is disposed on device body 18 and associated with control circuit 21 whereby control circuit 21 is further configured to allow occupant 111 of workstation 310 to functionally activate, deactivate, and/or modulate the intensity of said emanations 36 via operator interface 24. Alternatively, it is also conceived that operator interface 24 may be disposed at work surface 112 in a position easily accessible to workstation occupant 111 and be configured to communicate with control circuit 21 via a wired connection, said wired connection conceivably configured with one or more pluggable quick-connect/quick-disconnect connector(s) and port(s); however, wireless communication between operator interface 24 and control circuit 21 is also contemplated. At least the outer exposed surface of operator interface 24 may be comprised of copper or another material known to inactivate pathogens. Otherwise, operator interface 24 may be a proximity sensing hands-free interface. Furthermore, sensor 28 is disposed on the underside of work surface 112, said sensor 28 configured to sense the presence of workstation occupant 111. Sensor 28 is further associated with sensor cable 27 and in communication with controller 21 via said sensor cable 27 whereby the presence or absence of workstation occupant 111 is communicated to control circuit 21; however, wireless communication between operator sensor 28 and control circuit 21 is also contemplated. It is further contemplated that sensor 28 and sensor cable 27 may be further configured with one or more pluggable quick-connect/quick-disconnect connector(s) and port(s). Otherwise, sensor 28 may be disposed on or in device body 18.

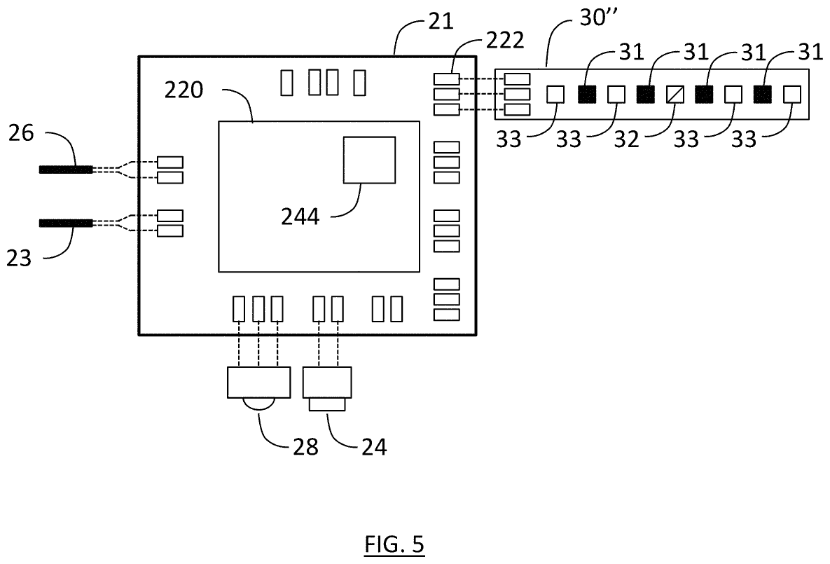

Referring now to FIG. 5, where control circuit 21 and I/O processor 220 are associated with disinfection energy sources 31 and light source 32 disposed on emitter base 30″, are separately associated with light sources 33 likewise disposed on emitter base 30″, and are additionally associated with user interface 24 and sensor 28, I/O processor 220 is configured to activate and/or deactivate disinfection energy sources 31 and light source 32 in unison in response to signals received via control cable 23 and/or motion sensor 28 and to activate, deactivate, and/or modulate the intensity of light sources 33 according to signals received via control cable 23, operator interface 24, and/or via motion sensor 28. Furthermore, in this embodiment, and accordance with the present invention, I/O processor 220 and timer circuit 244 are configured such that, when a signal for disinfection is received or otherwise present at control circuit 21 via control cable 23, I/O processor 220 and timer circuit 244 act to intermittently and simultaneously inversely modulate the intensity of light sources 33 and the intensity of disinfection energy sources 31 and light source 32 operating in unison so as to provide visual indication that a routine delivering disinfection emanations 34 and 34′ to office 300 and to workstation 310 is active (see FIG. 4) without exceeding the energy consumption of disinfection energy sources 31 and light source 32 operating alone.

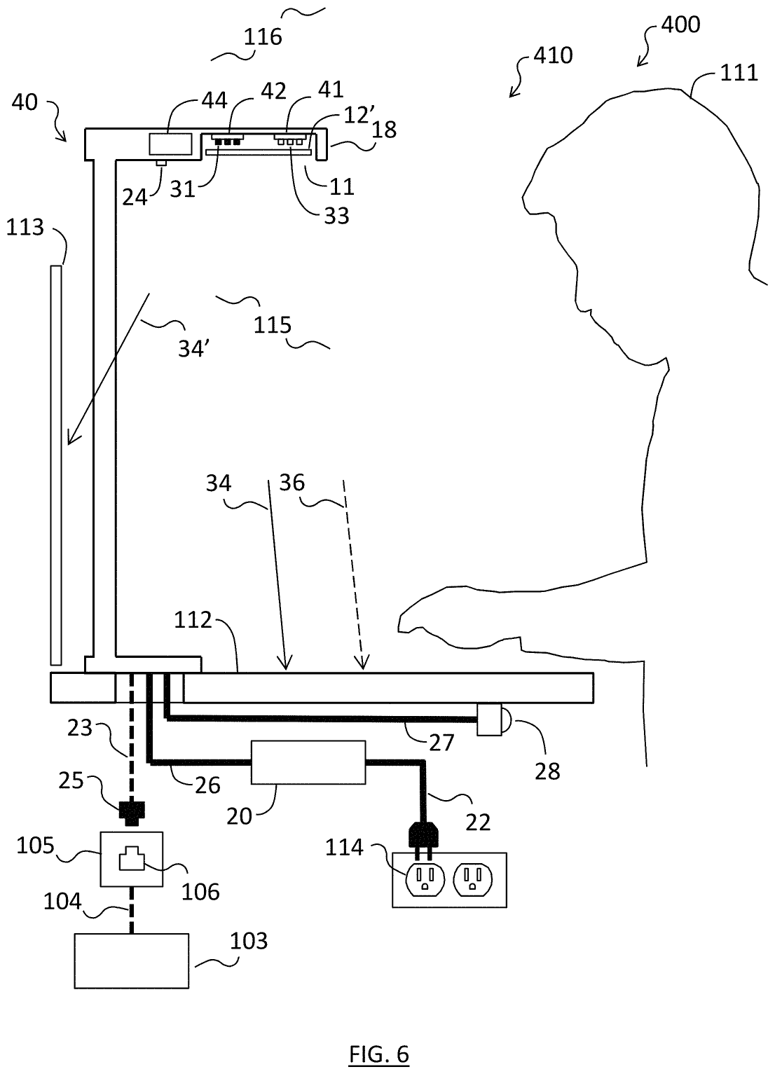

Referring now to FIG. 6, in another embodiment, where many of the items disclosed in office 300, workstation 310, and disinfection unit 10″ of FIG. 4 are deployed in office 400, workstation 410, and disinfection unit 40 disposed therein, disinfection energy source 31 is disposed on emitter base 42 and light source 33 is disposed on emitter base 41 such that emanations 34 and 34′ from disinfection energy source 31 pass through lens 12′ to provide for disinfection of work surface 112 and sneeze barrier 113, and any workstation accessories, office supplies, and/or personal effects disposed thereon, along with the air 115 circulating at workstation 410, and such that emanations 36 from light source 33 pass through lens 12′ to provide for task illumination of work surface 112. Disinfection unit 40 further comprises control circuit 44 associated with disinfection energy source 31 and light source 33. Control circuit 44 is further associated with power receptacle 114 via power cable 26, control unit 20, and cord and plug 22, and is in communication with controller 103 by means of control cable 23, pluggable connector 25, wiring terminal 105 comprising communication port 106, and control wiring 104, or by another means, whereby control circuit 40 is advantageously configured with a program to activate and deactivate disinfection energy source 31 and light source 33 in response to signals received from controller 103 and to advantageously activate, deactivate, and/or modulate the intensity of light sources 33 according to signals received via operator interface 24 and/or via motion sensor 28.

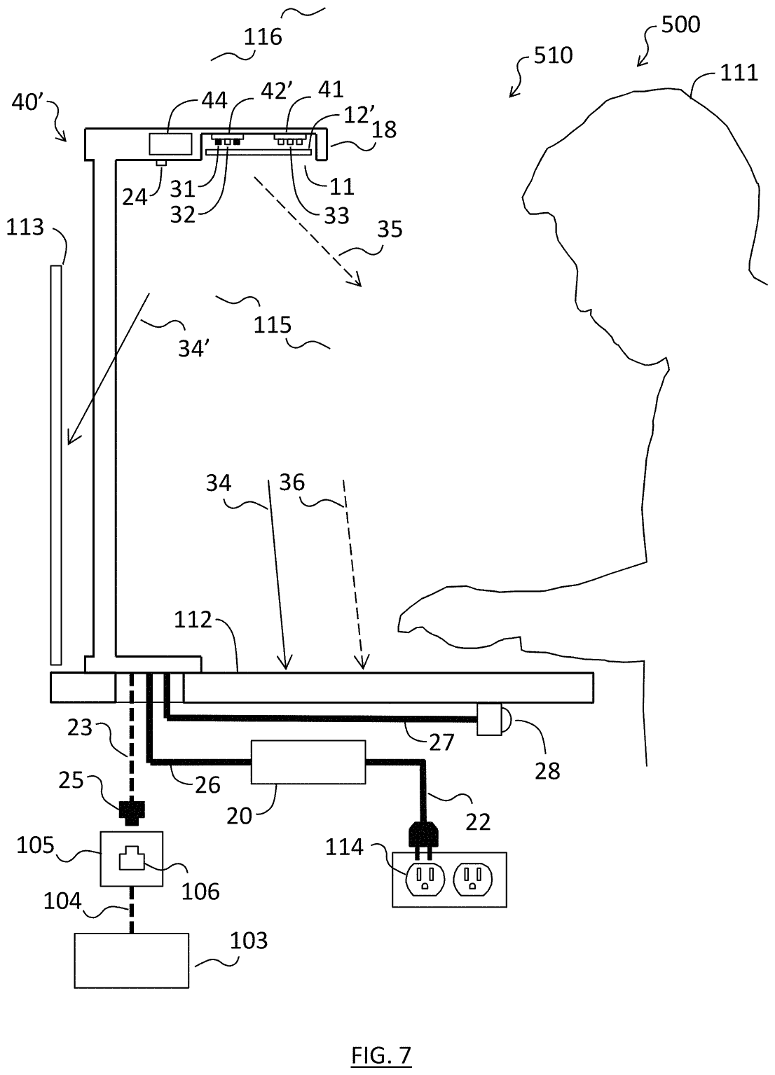

Referring now to FIG. 7, in another embodiment, where many of the items disclosed in office 400, workstation 410, and disinfection unit 40 of FIG. 6 are deployed in office 400, workstation 410, and disinfection unit 40′ disposed therein, disinfection energy source 31 and light source 32 are disposed on emitter base 42′ such that emanations 34 and 34′ from disinfection energy source 31 pass through lens 12′ to provide for disinfection of work surface 112 and sneeze barrier 113, and any workstation accessories, office supplies, and/or personal effects disposed thereon, along with the air 115 circulating at workstation 410, and such that emanations 35 from light source 32 pass through lens 12′ to generally provide for illumination of work surface 112 and other objects and surfaces occurring in workstation 210 and office 200 as seen by workstation occupant 111, other room occupants, and/or other persons viewing office 200 from any adjacent space. Accordingly, and in accordance with the present invention, light source 32 may be activated and deactivated in unison with disinfection energy source 31 and may further be one or more red LED(s) (i.e. an LED that emits light in the red region of the visual spectrum) whereby said illumination serves as a visual indicator that disinfection emanations 34 and 34′ are (or are not) present in office 500 and in workstation 510.

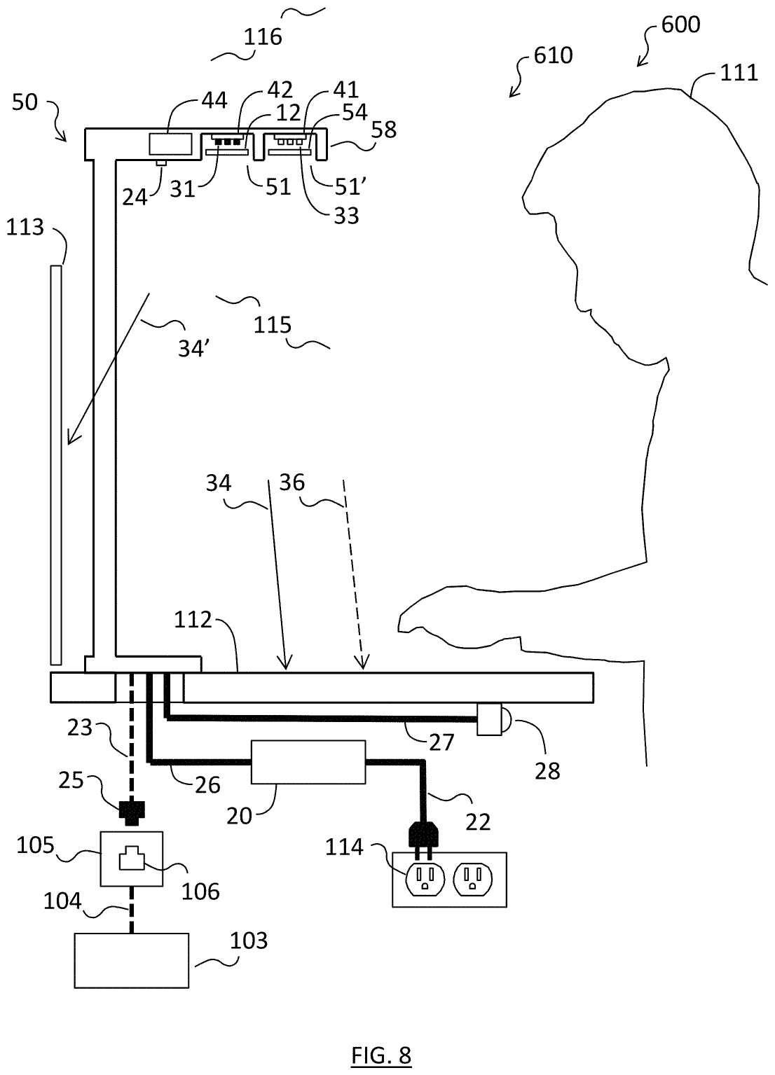

Referring now to FIG. 8, in another embodiment, where many of the items disclosed in office 100, office 400, office 500, workstation 100, workstation 410, workstation 510, disinfection units 10, 40, and 40′ of FIGS. 1, 5 and 6 are deployed in office 600, workstation 610, and disinfection unit 50 disposed therein, disinfection unit 50 further comprises emitter base 42 with disinfection energy source 31 disposed thereon, emitter base 41 with light source 33 disposed thereon, and device body 58, said device body 58 further comprising a first aperture 51 and a second aperture 51′. Furthermore, disinfection unit 50 comprises lens 54, said lens 54 further comprised of a material suitable for the transmission of light emitted by light source 33. In this embodiment, emitter base 42 and lens 12 are disposed in first aperture 51 such that emanations 34 and 34′ from disinfection energy source 31 pass through lens 12 to provide for disinfection of work surface 112 and sneeze barrier 113, and any workstation accessories, office supplies, and/or personal effects disposed thereon, along with the air 115 circulating at workstation 610. Likewise, emitter base 41 and lens 54 are disposed in second aperture 51′ such that emanations 36 from light source 33 pass through lens 54 to provide for to provide for task illumination of work surface 112.

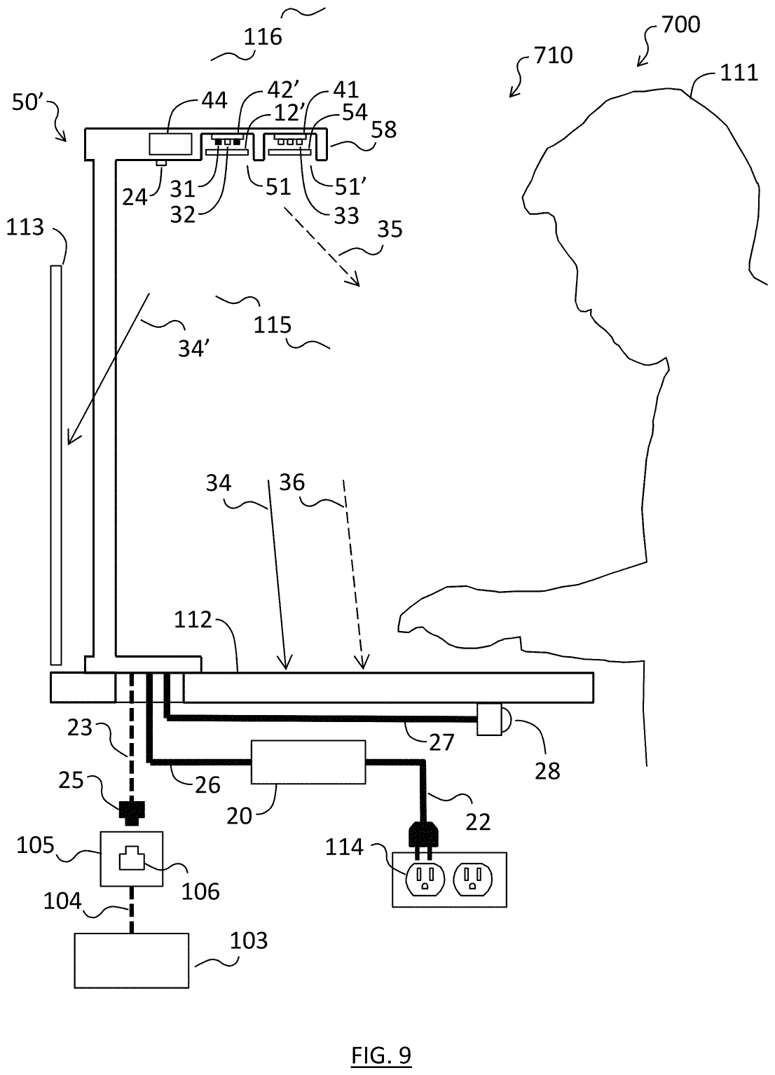

Referring now to FIG. 9, in another embodiment, where many of the items disclosed in office 500, office 600, workstation 510, and workstation 610, and disinfection unit 50 of FIGS. 7 and 8 are deployed in office 700, workstation 710, and disinfection unit 50′ disposed therein, emitter base 42′, with disinfection energy source 31 and light source 32 disposed thereon, and lens 12′ are disposed in said first aperture 51 such that emanations 34 and 34′ from disinfection energy source 31 pass through lens 12′ to provide for disinfection of work surface 112 and sneeze barrier 113, and any workstation accessories, office supplies, and/or personal effects disposed thereon, along with the air 115 circulating at workstation 410, and such that emanations 35 from light source 32 pass through lens 12′ to generally provide for illumination of work surface 112 and other objects and surfaces occurring in workstation 710 and office 700 as seen by workstation occupant 111, other room occupants, and/or other persons viewing office 700 from any adjacent space. Accordingly, and in accordance with the present invention, light source 32 may be activated and deactivated in unison with disinfection energy source 31 whereby said illumination serves as a visual indicator that disinfection emanations 34 and 34′ are (or are not) present in office 700 and in workstation 710.

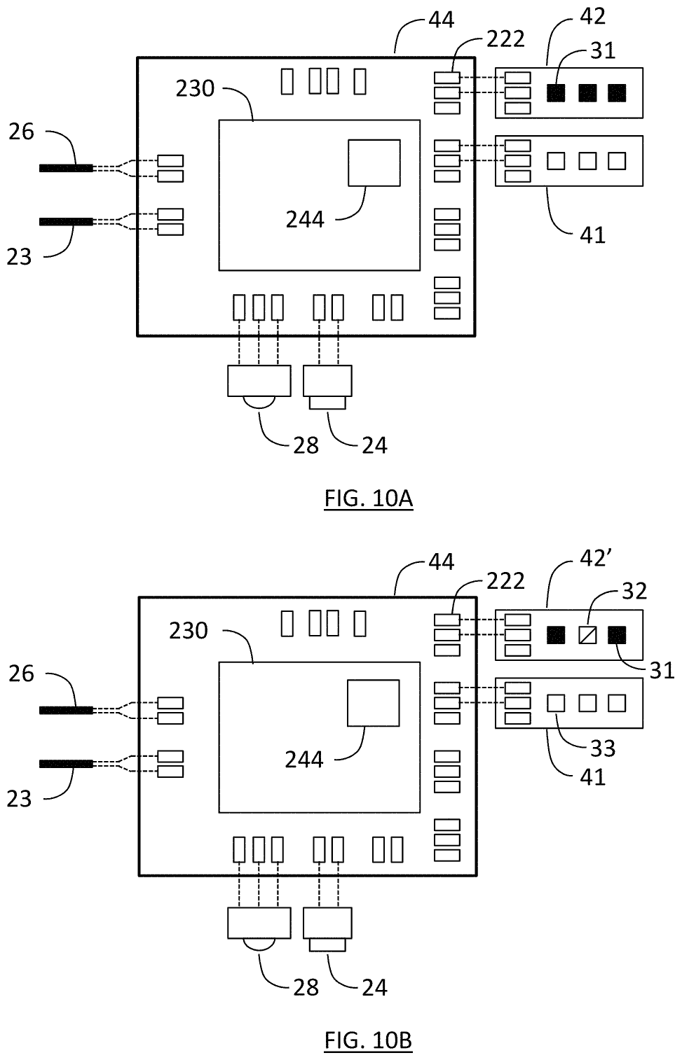

Referring now to FIGS. 10A and 10B, where emitter bases 41 with light sources 33 thereon, emitter base 42 with disinfection energy source 31 deployed thereon, and emitter base 42′ with light source 32 and disinfection energy source 31 together deployed thereon, are separately associated with control circuit 44 and I/O processor 230 along with user interface 24 and sensor 28, control circuit 44 and I/O processor 230 are configured to activate and/or deactivate disinfection energy sources 31 and light source 32 in unison on emitter base 42′ in response to signals received via control cable 23 and/or motion sensor 28, activate and/or deactivate disinfection energy sources 31 on emitter base 42 in response to signals received via control cable 23 and/or motion sensor 28, and activate, deactivate, and/or modulate the intensity of light sources 33 on emitter base 41 according to signals received via control cable 23, operator interface 24, and/or motion sensor 28. Furthermore, in this embodiment, and accordance with the present invention, I/O processor 230 and timer circuit 244 are configured such that, when a signal for disinfection is received or otherwise present at control circuit 44 via control cable 23, I/O processor 230 and timer circuit 244 act to intermittently and simultaneously inversely modulate the intensity of light sources 33 and the intensity of disinfection energy sources 31 and light source 32 operating in unison to provide visual indication that a routine delivering disinfection emanations 34 and 34′ is active (see FIGS. 6, 7, 8, and 9) without exceeding the energy consumption of disinfection energy sources 31 (combined with the energy consumption of light source 32, where occurs) operating alone.

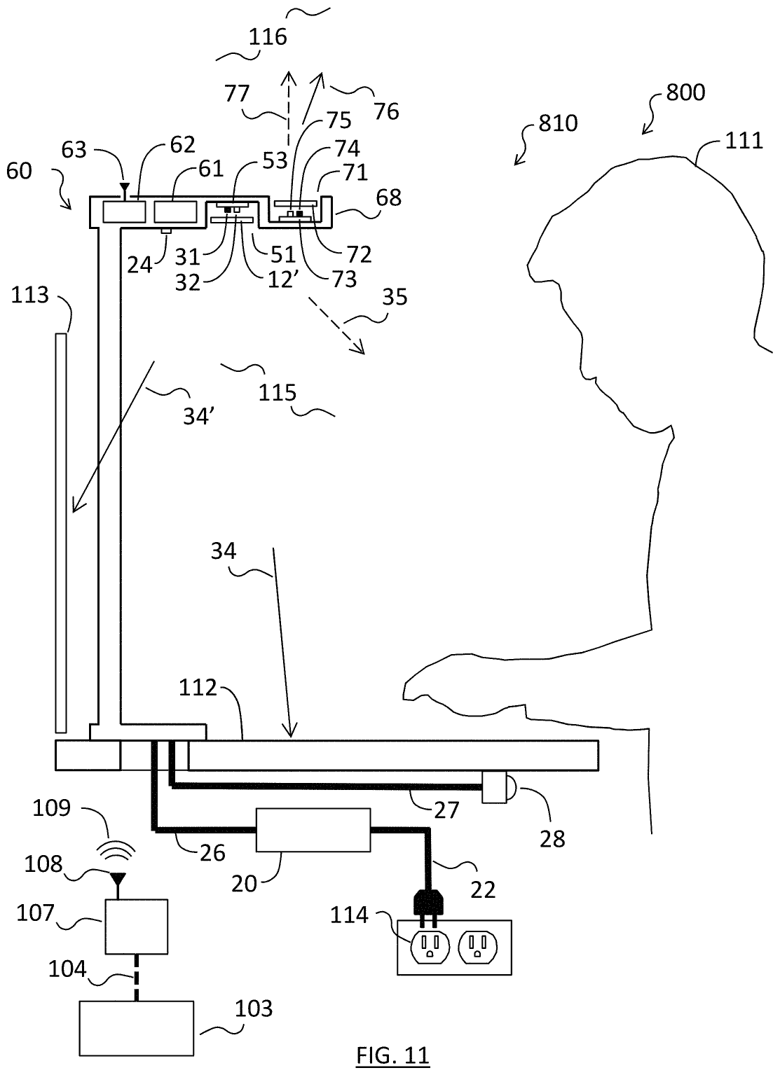

Referring now to FIG. 11, in another embodiment, where many of the items disclosed in office 700, workstation 710, and disinfection unit 50′ of FIG. 9 are deployed in office 800, workstation 810, and disinfection unit 60 disposed therein, disinfection unit 60 further comprises emitter base 53 with disinfection energy source 31 and light source 32 disposed thereon, emitter base 73 with disinfection energy source 74 and light source 75 disposed thereon, control circuit 61, wireless receiver 62, device body 68, said device body 68 further comprising a first aperture 51 and a second aperture 71, and lens 72, said lens 72 further comprised of a material suitable for the transmission of both light and electromagnetic energy of wavelengths emitted by disinfection energy source 74. In this embodiment, lens 12′ and emitter base 53, with disinfection energy source 31 and light source 32 disposed thereon, are disposed in said first aperture 51, such that emanations 34 and 34′ from disinfection energy source 31 pass through lens 12′ to provide for disinfection of work surface 112 and sneeze barrier 113, and any workstation accessories, office supplies, and/or personal effects disposed thereon, along with the air 115 circulating at workstation 810, and such that emanations 35 from light source 32 pass through lens 12′ to generally provide for illumination of work surface 112 and other objects and surfaces occurring in workstation 810 and office 800 as seen by workstation occupant 111, other room occupants, and/or other persons viewing office 800 from any adjacent space. Accordingly, and in accordance with the present invention, light source 32 may be activated and deactivated in unison with disinfection energy source 31 whereby said illumination serves as a visual indicator that disinfection emanations 34 and 34′ are (or are not) present in office 800 and in workstation 810.

Likewise, lens 72 and emitter base 73, with disinfection energy source 74 and light source 75 disposed thereon, are disposed in said second aperture 71, such that emanations 76 from disinfection energy source 74 pass through lens 72 to provide for disinfection of air 116 circulating above workstation 810, and such that emanations 77 from light source 75 pass through lens 72 to provide for general ambient illumination of office 800. Furthermore, disinfection energy source 74 may emit ultraviolet short-wave electromagnetic energy with a wavelength and/or intensity equal to that of disinfection energy source 31 or may emit ultraviolet short-wave electromagnetic energy of a different wavelength and/or intensity than that of disinfection energy source 31. Likewise, light source 75 may emit a white light spectral power distribution equal to that of light source 33 as disclosed in FIGS. 4, 5, 6, 7, and 8 for workstation task illumination or a different white light spectral power distribution.

Referring again to FIG. 11, transmitter 107 is disclosed in association with control wiring 104 such that transmitter 107 is in communication with controller 103. Furthermore, transmitter 107 comprises antenna 108, said antenna 108 configured to transmit wireless signals 109, and is disposed in or near office 800 and in wireless communication range of disinfection device 60 of workstation 810. Furthermore, control circuit 61 is in communication with receiver 62, said receiver 62 further comprising antenna 63 disposed on or in receiver 62 and configured to receive wireless signals 109. Antenna 63 may be disposed within device body 68 of disinfection device 60 or may extend through an opening in device body 68 to the exterior so as to improve wireless communication between transmitter 107 and receiver 62. Accordingly, control circuit 61 is in communication with controller 103 via wireless receiver 62, antenna 63, antenna 108, wireless transmitter 107 and control wiring 104. It is also contemplated that transmitter 107 and receiver 63 are transceivers such that receiver 62 is additionally configured to transmit wireless signals and transmitter 107 is additionally configured to receive said wireless signals. Furthermore, in this embodiment, control circuit 61 is associated with emitter base 53, with disinfection energy source 31 and light source 32 disposed thereon, and with emitter base 73, with disinfection energy source 74 and light source 75 disposed thereon, and is advantageously configured with a program to activate and deactivate disinfection energy source 31 and disinfection energy source 74 in response to signals originating at controller 103 and communicated wirelessly between transmitter 107 and receiver 62. Control circuit 61 may also be configured with a countdown timer whereby said program also activates said countdown timer in response to said signals originating at controller 103 and deactivates disinfection energy source 31 and disinfection energy source 74 when said countdown timer expires. In accordance with the present invention, transmitter 107 may transmit one or more wireless signal(s) to activate disinfection energy source 31 and disinfection energy source 74 when office 800 is known to be unoccupied and transmit one or more wireless signal(s) to deactivate disinfection energy source 31 and disinfection energy source 74 after a predetermined (time) period of disinfection and/or when occupancy of office 800 reoccurs. Furthermore, transmitter 107 may (re)transmit one or more wireless signal(s) to (re)activate disinfection energy source 31 and disinfection energy source 74 periodically as a signal heartbeat during each period of disinfection to reset said countdown timer and thereby prevent said countdown timer from expiring and deactivating disinfection energy source 31 and disinfection energy source 74 during said predetermined (time) period of disinfection. In a embodiment, said signal heartbeat may be configured to be 15 seconds and said countdown timer may be configured to expire in 20 seconds. In another embodiment, said signal heartbeat may be configured to be 120 seconds and said countdown timer may be configured to expire in 150 seconds, and in yet another embodiment, said signal heartbeat may be configured to be 120 seconds and said countdown timer may be configured to expire in 265 seconds. Accordingly, an advantageous automatic shut-OFF feature is provided to ensure that disinfection energy source 31 and disinfection energy source 74 do not remain active for long should one or more wireless deactivation signal(s) originating from controller 103 not be received by receiver 62.

Referring again to FIG. 11 it is further contemplated that controller 61 may be configured to receive power line carrier signals and to activate and deactivate disinfection energy source 31 and disinfection energy source 74 in response to signals transmitted by controller 103 to controller 61 by way of power receptacle 114, cord and plug 22, power supply unit 20, and power cable 26 or another wired connection between power supply unit 20 and controller 61.

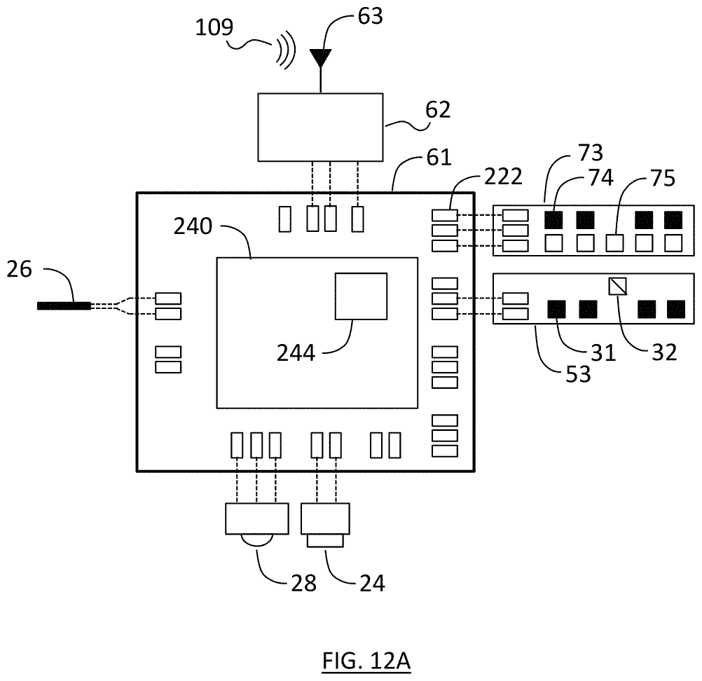

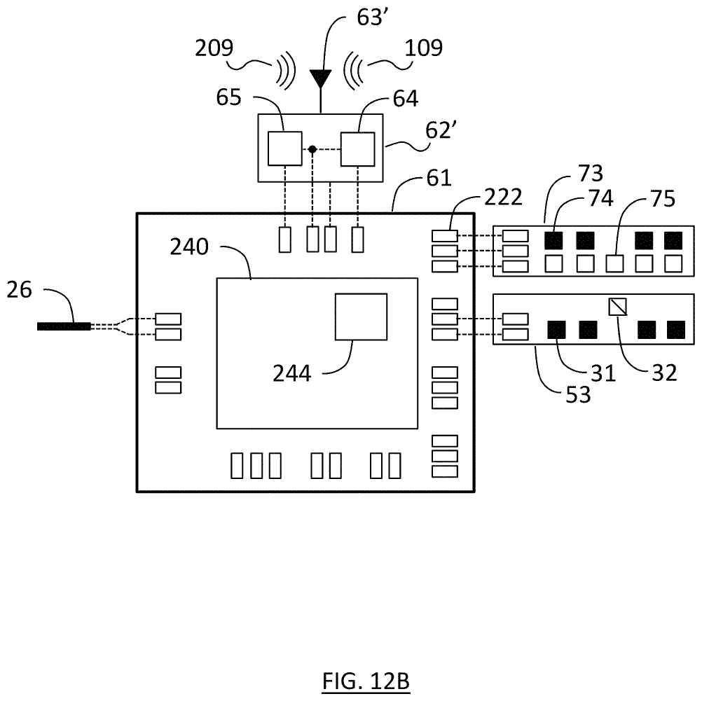

Referring now to FIG. 12A, where emitter base 53, with disinfection energy source 31 and light source 32 thereon, and emitter base 73, with disinfection energy source 74 and light source 75 deployed thereon, are separately associated with control circuit 61, said control circuit 61 comprising I/O processor 240 further comprising timer circuit 244, said timer circuit 244 further configured with a countdown timer program. Additionally, control circuit 61 is in communication with operator interface 24, sensor 28, and receiver 62, said receiver 62 comprising antenna 63 configured to receive wireless signals 109. Furthermore, in this embodiment, and accordance with the present invention, I/O processor 240 is configured such that, when receiver 62 receives a first wireless signal for disinfection, control circuit 61 activates disinfection energy source 31, disinfection energy source 74 and light source 32 and starts said countdown timer program of timer circuit 244. Furthermore, when said countdown timer program expires, control circuit 61 deactivates disinfection energy source 31, disinfection energy source 74, and light source 32, and resets said countdown timer program. Furthermore, when receiver 62 receives a second wireless signal for disinfection, control circuit 61 resets and (re)starts said countdown timer program of timer circuit 244 and (re)activates disinfection energy source 31, disinfection energy source 74 and light source 32. Accordingly, if receiver 62 does not receive a second wireless signal for disinfection before said countdown timer program expires, control circuit 61 deactivates disinfection energy source 31, disinfection energy source 74, and light source 32, and resets said countdown timer program. Furthermore, control circuit 61 and I/O processor 240 are configured to activate, deactivate, and/or modulate the intensity of light sources 75 on emitter base 73 according to signals received via operator interface 24 and/or via motion sensor 28.

Referring now to FIG. 12B, in another embodiment, where many of the items disclosed in disinfection unit 60 of FIG. 11 are associated with control circuit 61, control circuit 61 is further associated with receiver 62′, said receiver 62′ being a 2-channel control device comprising a first channel receiver 64, a second channel receiver 65, and at least one antenna 63′ in association with said first channel receiver and said second channel receiver, said antenna configured to receive wireless signals 109 and wireless signals 209. It is further conceived that wireless signals 209 may originate at transmitter 107 of office 800 (see FIG. 11) in response to control signals received by transmitter 107 from controller 103 or from another controller, or may originate at another transmitter associated with another controller. Furthermore, in this embodiment, and accordance with the present invention, control circuit 61 and I/O processor 240 are configured such that, when said first channel receiver 64 receives a first wireless signal for disinfection via antenna 63′, control circuit 61 activates disinfection energy source 31, disinfection energy source 74 and light source 32 and starts said countdown timer program of timer circuit 244. Furthermore, when said countdown timer program expires, control circuit 61 deactivates disinfection energy source 31, disinfection energy source 74, and light source 32, and resets said countdown timer program. Furthermore, when said first channel receiver 64 receives a second wireless signal for disinfection, control circuit 61 resets and (re)starts said countdown timer program of timer circuit 244 and (re)activates disinfection energy source 31, disinfection energy source 74, and light source 32. Accordingly, if said first channel receiver 64 does not receive a second wireless signal for disinfection before said countdown timer program expires, control circuit 61 deactivates disinfection energy source 31, disinfection energy source 74 and light source 32, and resets said countdown timer program. Furthermore, control circuit 61 and I/O processor 240 are configured to activate, deactivate, and/or modulate the intensity of light sources 75 on emitter base 73 in response to signals received via operator interface 24, and/or via motion sensor 28, and/or in response to wireless signals received by said first channel receiver and/or said second channel receiver via antenna 63′.

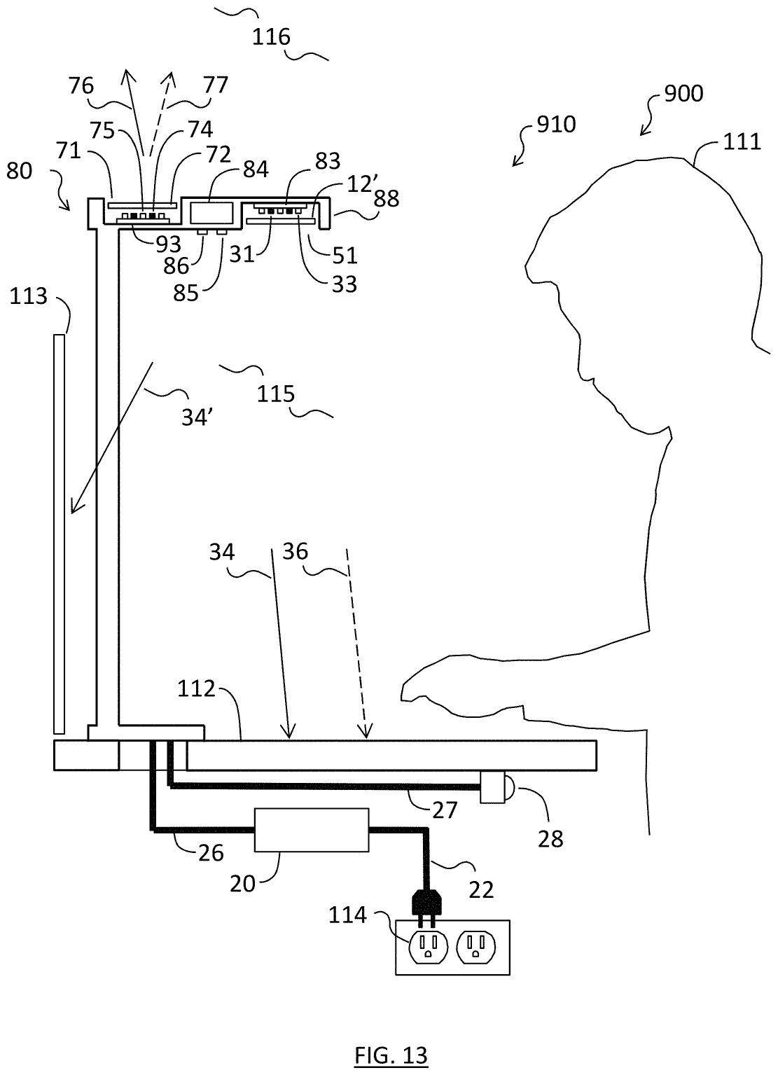

Referring now to FIG. 13, in another embodiment, where many of the items disclosed in office 800, workstation 810, and disinfection unit 60 of FIG. 9 are deployed in office 900, workstation 910, and disinfection unit 80 disposed therein, disinfection unit 80 further comprises emitter base 83, with disinfection energy source 31 and light source 32 disposed thereon, emitter base 93, with disinfection energy source 74 and light source 75 disposed thereon, device body 88, control circuit 84, a first operator interface 85, a second operator interface 86, and device body 88, said device body 88 further comprising a first aperture 51 and a second aperture 71. In this embodiment, emitter base 83, with disinfection energy source 31 and light source 33 disposed thereon, and lens 12′ are disposed in a first aperture 51 of device body 88 such that emanations 34 and 34′ from disinfection energy source 31 pass through lens 12′ to provide for disinfection of work surface 112 and sneeze barrier 113, and any workstation accessories, office supplies, and/or personal effects disposed thereon, along with the air 115 circulating at workstation 910, and such that emanations 36 from light source 33 pass through lens 12′ to provide for task illumination of work surface 112.

Likewise, emitter base 93, with disinfection energy source 74 and light source 75 disposed thereon, and lens 72 are disposed in said second aperture 71, such that emanations 76 from disinfection energy source 74 pass through lens 72 to provide for disinfection of air 116 circulating above workstation 910, and such that emanations 77 from light source 75 pass through lens 72 to provide for general ambient illumination of office 900. Furthermore, in this embodiment, disinfection energy source 31 and disinfection energy source 74 advantageously emit short-wave electromagnetic energy that is known to be harmless to humans and known to kill or otherwise deactivate pathogens such that disinfection device 80, and specifically disinfection energy source 31 and disinfection energy source 74 thereof, can be safely operated when office 900 and/or workstation 910 is occupied.

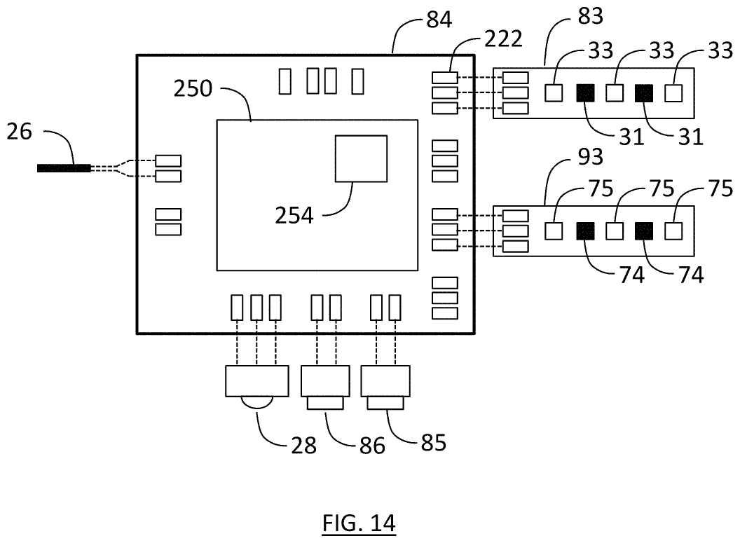

Referring now to FIG. 14, where many of the items disclosed in disinfection unit 80 of FIG. 13 are associated with control circuit 84, control circuit 84 comprises I/O processor 250, said I/O processor further comprising timer circuit 254, said timer circuit 254 configured with a countdown timer program. Furthermore, in this embodiment, control circuit 84 and I/O processor 250 are associated with disinfection energy sources 31 disposed on emitter base 83, are separately associated with light sources 33 likewise disposed on emitter base 83, are separately associated with disinfection energy sources 74 disposed on emitter base 93, and are separately associated with light sources 75 likewise disposed on emitter base 93. Furthermore, and in accordance with the present invention, control circuit 84 and I/O processor 250 are configured to activate, deactivate, and/or modulate the intensity of light sources 33 on emitter base 83 according to signals received via operator interface 85 and/or via motion sensor 28. Likewise, control circuit 84 and I/O processor 250 are configured to activate and deactivate disinfection sources 31 disposed on emitter base 83 and disinfection energy sources 74 disposed on emitter base 93 in unison according to signals received via operator interface 86. Furthermore, control circuit 84 and I/O processor 250 are configured to commence said countdown timer program upon activation of disinfection energy sources 31 and 74, to terminate and reset said countdown timer program upon deactivation of disinfection energy sources 31 and 74, and to automatically deactivate disinfection energy sources 31 and 74 and reset said countdown timer program when said countdown timer program expires.

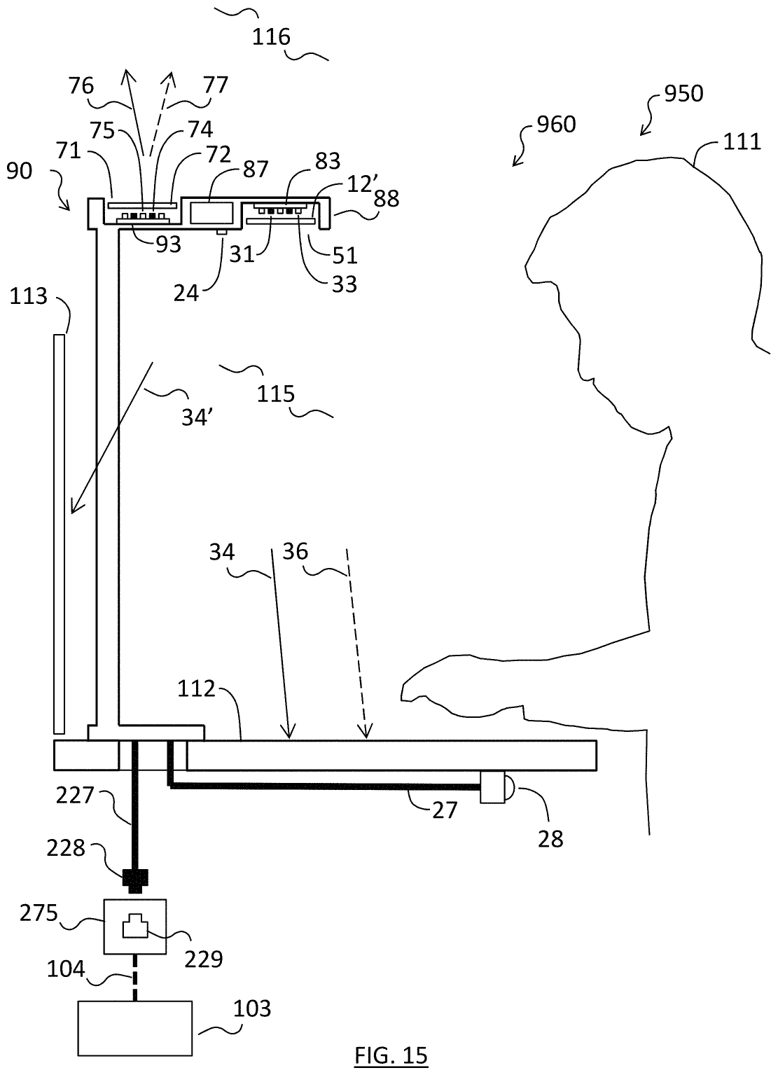

Referring now to FIG. 15, in another embodiment, where many of the items disclosed in office 900, workstation 910, and disinfection unit 90 of FIG. 13 are deployed in office 950, workstation 960, and disinfection unit 90 disposed therein, disinfection unit 90 further comprises control circuit 87 and multi-conductor cable 227 comprising quick-connect/quick-disconnect pluggable connector 228. Furthermore, in this embodiment, office 950 further comprises power and data switch 275 disposed in proximity to workstation 112 and comprising quick-connect/quick-disconnect pluggable port 228. Furthermore, in this embodiment, disinfection energy source 31 and disinfection energy source 74 advantageously emit short-wave electromagnetic energy that is known to kill or otherwise deactivate pathogens. Accordingly, energy emitted by disinfection energy source 31 and disinfection energy source 74 may have a peak intensity wavelength between 200 nm and 420 nm. It is also contemplated that the peak intensity emanations of energy source 31 and/or disinfection energy source 74 may comprise other wavelengths of short-wave electromagnetic energy known to kill or otherwise deactivate pathogens.

Referring again to FIG. 15, said multi-conductor cable 227 with quick-connect/quick-disconnect pluggable connector 228 is in association with control circuit 87 and may also be in association with disinfection energy source 31 and disinfection energy source 74 such that electrical power and/or communication signals, such as digital communications signals originating at controller 103, are delivered and communicated to control circuit 87 and/or to disinfection energy source 31 and disinfection energy source 74. Furthermore, said multi-conductor cable 227 with quick-connect/quick-disconnect pluggable connector 228 may be a Power-over-Ethernet cable.

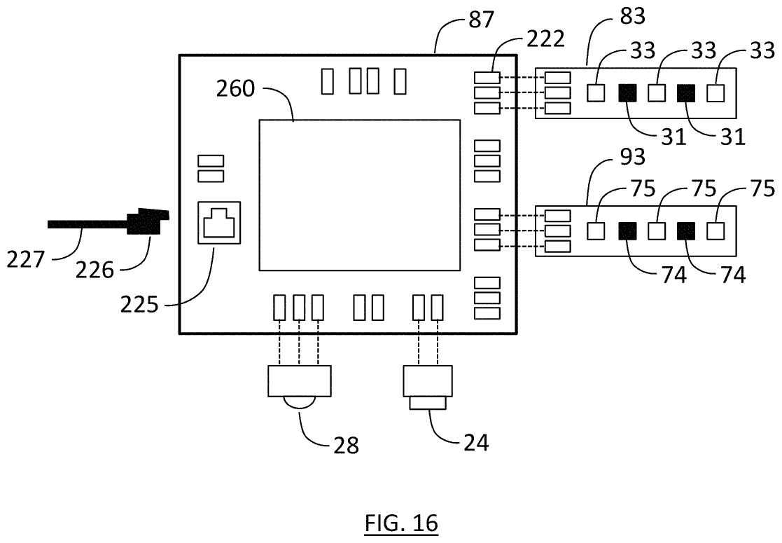

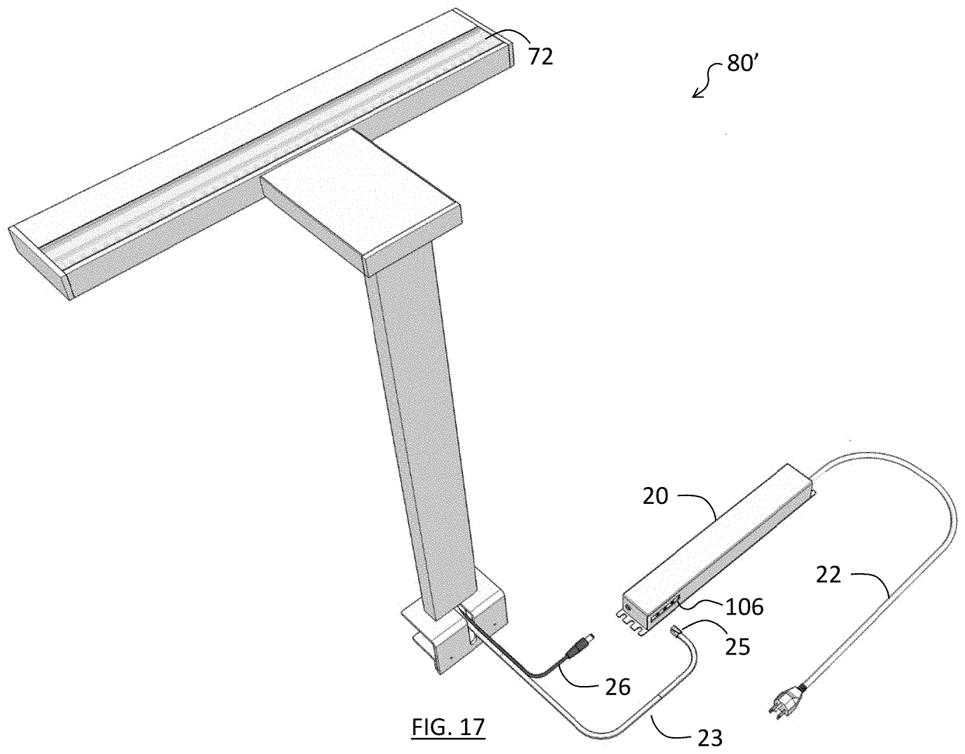

Referring now to FIG. 16, where many of the items disclosed in disinfection unit 90 of FIG. 15 are associated with control circuit 87, control circuit 87 comprises I/O processor 260. Furthermore, in this embodiment, control circuit 87 and I/O processor 260 are associated with disinfection energy sources 31 disposed on emitter base 83, are separately associated with light sources 33 likewise disposed on emitter base 83, are separately associated with disinfection energy sources 74 disposed on emitter base 93, and are separately associated with light sources 75 likewise disposed on emitter base 93. Furthermore, control circuit 87 and I/O processor 260 are configured to activate, deactivate, and/or modulate the intensity of light sources 33 on emitter base 83 according to signals received via multi-conductor cable 227 and/or via operator interface 85 and/or via motion sensor 28. Likewise, control circuit 87 and I/O processor 260 are configured to activate and deactivate disinfection sources 31 disposed on emitter base 83 and disinfection energy sources 74 disposed on emitter base 93 in unison or separately according to signals received via multi-conductor cable 227 and/or via motion sensor 28. It is also contemplated that any one or more of disinfection sources 31, disinfection sources 74, light sources 33, and light sources 75. may be in direct association with controller 103 (see FIG. 15) and that controller 103 may be configured such that said controller 103 may activate, deactivate, and/or modulate the intensity of any one or more of said disinfection sources 31, disinfection sources 74, light sources 33, and light sources 75.

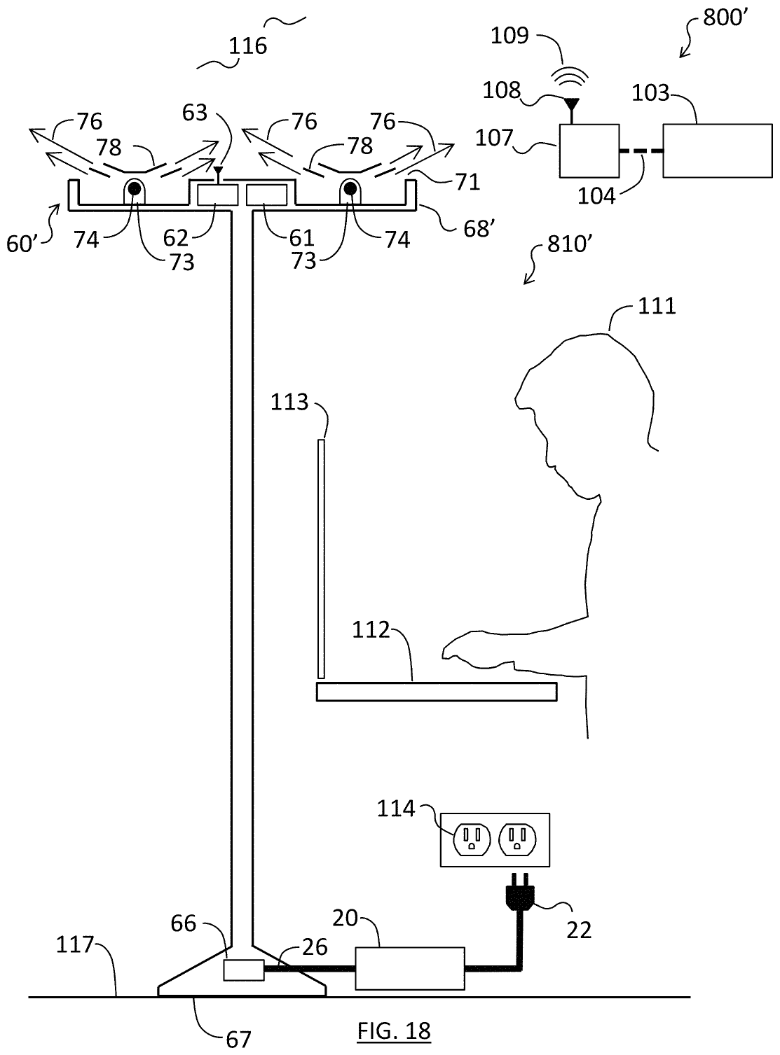

Referring now to FIG. 18, in another preferred embodiment, where many of the items disclosed in office 800, workstation 810, and disinfection unit 60 of FIG. 11 are deployed in office 800′, workstation 810′, and disinfection unit 60′ disposed therein. Disinfection unit 60′ is disposed on mounting surface 117 of workstation 810′ and comprises emitter bases 73 with associated disinfection energy sources 74, control circuit 61, wireless receiver 62, and device body 68′, said device body 68′ further comprising a first aperture 71 and louvers 78, said louvers 78 configured to direct disinfection emanations 76 emitted by disinfection energy source 74 upwardly and away from workstation occupant 111, or to shield workstation occupant 111 and other room occupants from said disinfection emanations, or both. In this preferred embodiment, louver 78 and emitter base 73, with disinfection energy source 74 associated therewith, are disposed in said first aperture 71, such that disinfection emanations 76 from disinfection energy source 74 pass through openings in louver 78 to provide for disinfection of the air 116 circulating above workstation 810′ and above workstation occupant 111. In an exemplary embodiment, disinfection energy source 74 may be a tubular low-pressure mercury discharge lamp that emits short-wave electromagnetic energy that is known to kill or otherwise deactivate pathogens. Accordingly, energy emitted by disinfection energy source 74 and disinfection energy source may have a peak intensity wavelength of 254.3 nm. It is also contemplated that the emanations of disinfection energy source 74 may comprise other wavelengths of short-wave electromagnetic energy known to kill or otherwise deactivate pathogens.

Referring again to FIG. 18, transmitter 107 is disclosed in association with control wiring 104 such that transmitter 107 is in communication with controller 103. Furthermore, transmitter 107 comprises antenna 108, said antenna 108 configured to transmit wireless signals 109, and is disposed in or near office 800′ and in wireless communication range of disinfection device 60′ of workstation 810′. Furthermore, control circuit 61 is in communication with receiver 62, said receiver 62 further comprising antenna 63 disposed on or in receiver 62 and configured to receive wireless signals 109. Accordingly, control circuit 61 is in communication with controller 103 via wireless receiver 62, antenna 63, antenna 108, wireless transmitter 107 and control wiring 104. Furthermore, in this embodiment, control circuit 61 is associated with disinfection energy source 74 via emitter base 73 and is advantageously configured with a program to activate and deactivate disinfection energy source 74 in response to signals originating at controller 103 and communicated wirelessly between transmitter 107 and receiver 62.

In accordance with the present invention, transmitter 107 may transmit one or more wireless signal(s) to activate disinfection energy source 74 when office 800′ is known to be occupied and transmit one or more wireless signal(s) to deactivate disinfection energy source 74 when office 800′ is vacant. Furthermore, in a preferred embodiment wherein disinfection body 68′ comprises mounting face 67 and shut-off switch 66, and wherein mounting surface 117 is a floor or another horizontal surface of workstation 810′, shut-off switch 66 is configured to associate controller 61 with power cable 26 when mounting face 67 of disinfection body 68′ is disposed on mounting surface 117 and to disassociate controller 61 from power cable 26 when disinfection surface 67 is not disposed thereon so as to advantageously prevent disinfection energy source 74 from emitting disinfection emanations 76 when device 60′ is not in association with mounting surface 117. Similarly, shut-off switch 66 may be a tilt-switch that serves to deactivate disinfection energy source 74 when mounting face 67 is disposed at a range of angles to mounting surface 117 (e.g. all angles greater than 5 degrees). In another contemplated embodiment of the invention, shut-off switch 66 is a tilt-switch circuit comprised integral to control circuit 61.

Referring now to FIG. 3D, it is further contemplated that, in the various embodiments presented herein, power cable 26 may comprise quick-connect/quick-disconnect pluggable connector 94 and control circuits 21, 44, 61 and 84 may comprise corresponding quick-connect/quick-disconnect pluggable port 95 whereby power cable 26 is associated with control circuits 21, 44, 61 and 84 via said quick-connect/quick-disconnect pluggable connector and port. Likewise, it is further contemplated that, in the various embodiments presented herein, control cable 23 may comprise quick-connect/quick-disconnect pluggable connector 96 and control circuits 21, 44, 61 and 84 may comprise corresponding quick-connect/quick-disconnect pluggable port 97 whereby control cable 23 is associated and communicates with control circuits 21, 44, 61 and 84 via said quick-connect/quick-disconnect pluggable connector and port. It is also contemplated that any or all of emitter bases 30, 30′, 30″, 41, 42, 42′, 53, 73, 83 and 93, operator interfaces 24, 85 and 86, sensor 28, and receivers 62 and 62′ may be associated and/or may communicate with control circuits 21, 44, 61 and 84 via similar quick-connect/quick-disconnect pluggable connector(s) and port(s).

Above, with reference to FIG. 12A, the control circuit 21 was described as comprising the timer circuit 244. This configuration is of course exemplary. The broad scope of the disclosure contemplates the timer circuit 244 being disposed at any location sufficient for performing its intended function as described herein. For example, in the embodiment of FIG. 12A, the receiver 62 may comprise the timer circuit 244 configured with said countdown timer program thereof. Similarly, in other embodiments disclosed herein, the various described receivers may comprise such a timer circuit. Overall, the descriptions provided herein are general in nature, and various components of the plurality of embodiments may be exchanged and/or repositioned within and amongst the various embodiments in accordance with the present disclosure.

While the disclosure has been described with reference to a embodiment, it will be understood by those skilled in the art that various changes may be made and equivalents may be substituted for elements thereof without departing from the scope of the disclosure. In addition, many modifications may be made to adapt a particular situation or material to the teachings of the disclosure without departing from the essential scope thereof. Therefore, it is intended that the disclosure not be limited to the particular embodiment disclosed as the best mode contemplated for carrying out this disclosure, but that the disclosure will include all embodiments falling within the scope of the appended claims. Moreover, the use of the terms first, second, etc. do not denote any order or importance, but rather the terms first, second, etc. are used to distinguish one element from another.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A disinfection device for a workstation disposed in an office, said disinfection device comprising: at least one disinfection energy source, a cord and plug configured for removable association with a power receptacle of said workstation, and at least one control circuit configured to activate and deactivate said disinfection energy source.

2. The disinfection device of claim 1 wherein said control circuit is in communication with a remote controller of said office.

3. The disinfection device of claim 2 wherein said remote controller operates via automatic control and said at least one control circuit is configured to operate said disinfection energy source in a corresponding automatic manner.

4. The disinfection device of claim 1 wherein said control circuit comprises a countdown timer program.

5. The disinfection device of claim 1 wherein said disinfection device comprises at least one light source.

6. The disinfection device of claim 5 wherein said disinfection device comprises at least one operator interface for controlling said at least one light source.

7. The disinfection device of claim 5 wherein said control circuit is in communication with a remote controller of said office.
